(19) United States
(12) Patent Application Publication
(10) Pub. No.: US 2025/0090063 A1
(43) Pub. Date: Mar. 20, 2025

(54) BLADDER MONITORING METHOD AND APPARATUS BASED ON MULTI-CHANNEL STRAIN BLADDER SENSOR, AND MULTI-CHANNEL STRAIN BLADDER SENSOR VERIFICATION METHOD AND APPARATUS

(71) Applicant: DAEGU GYEONGBUK INSTITUTE OF SCIENCE AND TECHNOLOGY, Daegu (KR)

(72) Inventors: Sang Hoon LEE, Daegu (KR); Hee Jae SHIN, Daegu (KR); Young Jun CHO, Daegu (KR); Yu Jin JO, Daegu (KR)

(21) Appl. No.: 18/841,465

(22) PCT Filed: Apr. 19, 2023
(86) PCT No.: PCT/KR2023/005276

§ 371 Date: Aug. 26, 2024

(30) Foreign Application Priority Data
Feb. 28, 2022 (KR) 10-2022-0026338
May 26, 2022 (KR) 10-2022-0064876

(51) Int. Cl. A61B 5/20

(52) U.S. Cl. CPC A61B 5/202

(57) ABSTRACT

Disclosed are a bladder monitoring method and apparatus based on a multi-channel strain bladder sensor, and a multi-channel strain bladder sensor verification method and apparatus. The multi-channel strain bladder sensor verification method according to an embodiment of the present disclosure may comprise the steps of: controlling expansion and contraction of a measurement object acting as a bladder; acquiring a sensing value from each channel of a bladder sensor attached to the measurement object, according to the control of expansion and contraction of the measurement object; tracking the volume change and expansion direction of the measurement object by analyzing data regarding the change in the sensing value from each channel of the bladder sensor; and verifying the bladder sensor on the basis of whether the volume change and expansion direction of the measurement object are included within the standard range corresponding to the control of expansion and contraction of the measurement object.

### TECHNICAL FIELD

The present disclosure relates to a bladder monitoring method and apparatus based on a multi-channel strain bladder sensor to monitor the condition of the bladder, by using a multi-channel strain bladder sensor for tracking anisotropic volume changes of the bladder, and to a multi-channel strain bladder sensor verification method and apparatus.

### BACKGROUND ART

Bladder diseases are one type of the newly emerging issues in an aging society, and many people are suffering from bladder diseases all over the world. Recently, the neuro-stimulation technique has been attracting attention as one of the methods for treating bladder diseases. However, the application of such a technique requires a method for effectively monitoring the bladder condition to identify the appropriate timing for stimulation.

For this, there have been studied various implantable and non-invasive sensors for monitoring the bladder condition. However, a considerable number of studies did not fully comply with the bladder's characteristics requiring high elasticity. Moreover, few studies have observed bladder movements through monitoring with sensors.

Bladder dysfunction is mostly associated with impaired nerve function called neurogenic bladder, which includes urinary issues like inability to control urination. There are many factors causing neurogenic bladder, such as illness and injury. For example, patients with spinal cord injuries may suffer from bladder dysfunction temporarily or permanently, making it difficult for the patients to live normally. Another issue with bladder dysfunction is that other diseases may developed if urination is not timely.

Thus, many studies are still needed to return patients to normal lives although hospitals provide some practical solutions. Medical staff, bioengineers, and neuro-engineers are exerting considerable effort to resolve bladder dysfunction, and biotechnology companies are showing interest in the issue.

One of the promising solutions, especially for underactive bladder, is to implant a neurostimulator into the body and induce micturition through stimulation relevant to bladder nerves. However, due to the damage to sensory neurons in the bladder, patients are not able to sense the moment when bladder is full. Therefore, a monitoring system for measuring the bladder condition is required to apply the timely treatment using neuro-stimulation.

Meanwhile, there are typically two ways to measure bladder pressure. The first one is to implant a sensor inside the bladder or the bladder wall to measure internal pressure of the bladder. However, due to severe environment inside the bladder, such as the presence of urine and sharp pressure changes, the packaging of the sensor to withstand chemical and physical changes is one of the challenging issues. Recording reliable pressures in the environment by such a sensor and transmitting output signals outside the body are also crucial challenges.

The second one is to attach a sensor on the surface of the bladder wall to measure volume changes of the bladder. For example, a stick-shaped sensor attached to the bladder wall stretches together with the expanding bladder, providing the volume change of the bladder through the output change of the sensor.

The bladder sensor used is a device that is designed to provide information about the bladder's degree of expansion to patients who cannot sense the timing of urination themselves due to abnormal bladder function caused by spinal or nerve damage, thus allowing the patients to determine the urination timing themselves.

There were many studies of sensors that monitor the bladder volume, and there were various types of sensors according to the operating mechanisms. The bladder sensors were studied with many sensing methods including resistive sensors, capacitive sensors, accelerometers, piezoelectric sensors, piezoresistive sensors, and triboelectric nanogenerators (TENGs). However, each of these methods has its own drawbacks.

First, capacitive sensors are difficult to apply to circuits since the output value is small depending on the strain. Also, one capacitive sensor showed a limited sensing volume ranges (100-200 ml) and an operation frequency range (8.5-9.5 MHz).

Additionally, the study on the implantation of an accelerometer and a piezoelectric sensor inside the bladder wall is likely to damage the bladder wall muscle, resulting in limited clinical applications. In addition, the method using TENGs requires special equipment for measuring the output signal, like an oscilloscope, due to the high internal impedance of the device, and a sufficient output signal is generated only when a target is largely mechanically moved.

Meanwhile, resistive sensors are suitable for volume monitoring of the bladder due to the unique characteristics of the bladder, and have a simple mechanism and are relatively easy to apply to a circuit. Therefore, resistive sensors, which have been studied the most among the various sensing methods, are devices that can indirectly track the degree of expansion of the bladder by checking the change in resistance value of the sensor as the outer wall of the bladder expands or contracts.

Most of the conventionally used sensors were proposed with very simple single-channel designs in order to respond to very large bladder changes after being fixed to the bladder surface, and were attached to specific positions to indirectly measure the amount of urine inside the bladder.

Since the bladder is a three-dimensional object and has varying degrees of expansion at different positions (sites), conventional stick-shaped sensors may measure different expansion rates depending on their attachment direction and position, resulting in limitations in reproducibility and information accuracy. Moreover, small-sized single-channel stick-shaped sensors are not sufficient to accurately measure the change of the bladder, of which the volume increases by three time or more.

Additionally, it is very important to identify the anisotropic movement of the bladder during urination since additional urination needs to be induced if the bladder is not completely emptied.

In order to track the anisotropic volume change of the bladder, data for all the directions [+x, −x, +y, −y] of expansion need to be acquired, but existing sensors cannot secure such data, making it difficult to track the anisotropic volume change of the bladder.

In other words, there is a need for stable and accurate verification to measure fabricated sensors in a closed-loop system environment with a urination system similar to that of the human body.

(Non-Patent Document 1) Prior Art Document 1: Hannah, S., Brige, P., Ravichandran, A., & Ramuz, M. “Conformable, stretchable sensor to record bladder wall stretch.” ACS Omega, 4(1), 2019, pp. 1907-1915.

(Non-Patent Document 2) Prior Art Document 2: Yan, D., Bruns, T. M., Wu, Y, Zimmerman, L. L., Stephan, C., Cameron, A. P. et al. “Ultra-compliant carbon nanotube direct bladder device.” Advanced healthcare materials, 8(20), 2019, pp. 1900477.

The background art described above may be technical information retained by the present inventors in order to derive the present disclosure or acquired by the present inventors along the process of deriving the present disclosure, and thus is not necessarily a known art disclosed to the general public before the filing of the present application.

### DISCLOSURE OF INVENTION

### Technical Problem

An aspect of the present disclosure is to monitor the bladder condition and provide more accurate information on urination timing by acquiring data in all directions of expansion of the bladder through a multi-channel strain bladder sensor for tracking anisotropic volume changes of the bladder.

Another aspect of the present disclosure is to improve the measurement accuracy and stability of a bladder sensor by performing bladder sensor verification in a closed-loop system environment with a urination system similar to that of the human body.

Objects of the present disclosure are not limited to the above-mentioned object, and other objects and advantages of the present disclosure, which are not mentioned, will be understood through the following description, and will become apparent from embodiments of the present disclosure. It is also to be understood that the objects and advantages of the present disclosure may be realized by means and combinations thereof set forth in claims.

### Solution to Problem

A multi-channel strain bladder sensor verification method according to the present disclosure may include: controlling the expansion and contraction of a measurement object serving as a bladder; obtaining sensing values from each channel of a bladder sensor attached to the measurement object according to the controlling of the expansion and contraction of the measurement object; tracking the volume change and expansion direction of the measurement object by analyzing change data in sensing values for each channel of the bladder sensor; and verifying the bladder sensor on the basis of whether the volume change and expansion direction of the measurement object are within reference ranges corresponding to the control of the expansion and contraction of the measurement object.

Additionally, there may be further provided other methods, other systems, and a computer-readable recording medium having a computer program stored thereon to execute the methods.

Other aspects, features, and advantages other than those described above will become apparent from the following drawings, claims, and detailed description of the present disclosure.

### Advantageous Effects of Invention

According to embodiments of the present disclosure, data in all directions of expansion of the bladder can be obtained by using a multi-channel strain bladder sensor for tracking anisotropic volume changes of the bladder, thereby monitoring the bladder condition and providing more accurate information on urination timing.

Furthermore, the degree of expansion of the bladder can be tracked regardless of the attachment location and direction of the sensor by enabling the multi-channel strain bladder sensor to track the volume change of the bladder as well as the direction of expansion of anisotropically expanding bladder muscles.

Furthermore, the development associated with the muscle wall surface and expansion mechanism of the bladder can be achieved by securing individual data on the degrees of expansion for different parts of the bladder.

Furthermore, the measurement accuracy and stability of the bladder sensor can be improved by performing bladder sensor verification in a closed-loop system environment with a urination system similar to that of the human body.

Furthermore, the stability in future clinical trials can be ensured by previously testing the performance of the sensor, which is to be exhibited in the actual body environment, through a closed-loop system before inserting the sensor into the body.

The advantages of the present disclosure are not limited to those mentioned above, and other advantages not mentioned will be clearly understood by a person skilled in the art from the following description.

### BRIEF DESCRIPTION OF DRAWINGS

FIG. 1 schematically shows a bladder sensor tracking system environment for performing bladder monitoring based on a multi-channel strain bladder sensor according to an embodiment.

FIG. 2 shows a fabrication process of a bladder sensor according to an embodiment.

FIG. 3 shows the electrodeposition of an AuCNT composite according to an embodiment.

FIG. 4 shows a cross-sectional view of a bladder sensor according to an embodiment.

FIG. 5 shows a design of a multi-channel bladder sensor according to an embodiment.

FIGS. 6 and 7 show the optimization of process conditions according to an embodiment.

FIG. 8 compares CNT bladder sensors and AuCNT-added bladder sensors according to an embodiment.

FIG. 9 illustrates a method for measuring the strain resistances of a stick-shaped bladder sensor according to an embodiment.

FIG. 10 is a block diagram schematically showing a bladder sensor tracking system for performing the verification of a multi-channel strain bladder sensor and the monitoring thereby according to an embodiment.

FIG. 11 schematically shows a verification unit for performing the bladder sensor verification according to an embodiment.

FIG. 12 is a flow chart for illustrating a multi-channel strain bladder sensor verification method according to an embodiment.

FIG. 13 illustrates the characteristics of a stick-shaped bladder sensor depending on the volume of a balloon model according to an embodiment.

FIG. 14 illustrates a verification experiment using a light emitter according to an embodiment.

FIG. 15 illustrates a verification experiment of a stick-shaped bladder sensor by using pig's bladder according to an embodiment.

FIG. 16 illustrates the volume resistance characteristics of a multi-channel bladder sensor according to an embodiment.

FIG. 17 shows the characteristics of the multi-channel bladder sensor used in the balloon model according to an embodiment.

FIG. 18 illustrates the volume resistance characteristics of a bladder sensor in pig's bladder according to an embodiment.

FIG. 19 shows the experimental results for detecting the expansion direction of a bladder sensor according to an embodiment.

FIG. 20 illustrates the bladder expansion depending on the volume of a balloon model according to an embodiment.

FIG. 21 illustrates a concept of detecting the expansion direction of the bladder through a multi-channel bladder sensor according to an embodiment.

FIG. 22 is a flow chart for illustrating a bladder monitoring method based on a multi-channel strain bladder sensor according to an embodiment.

### MODE FOR CARRYING OUT THE INVENTION

The advantages and features of the present disclosure and methods of achieving the same will be apparent from the embodiments described below in detail in conjunction with the accompanying drawings.

However, the description of particular exemplary embodiments is not intended to limit the present disclosure to the particular exemplary embodiments disclosed herein, but on the contrary, it should be understood that the present disclosure is to cover all modifications, equivalents, and alternatives falling within the spirit and scope of the present disclosure. The embodiments disclosed below are provided so that this disclosure will be thorough and complete and will fully convey the scope of the present disclosure to those skilled in the art. In the following description of embodiments of the present disclosure, a detailed description of related known technology will be omitted when the same may obscure the subject matter of the embodiments of the present disclosure.

The terminology used herein is used for the purpose of describing particular example embodiments only and is not intended to be limiting. It must be noted that, as used herein and in the appended claims, the singular forms include the plural forms unless the context clearly dictates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. Furthermore, terms such as “first,” “second,” and other numerical terms, are used only to distinguish one element from another element. These terms are generally only used to distinguish one element from another.

Hereinafter, embodiments of the present disclosure will be described in detail with reference to the accompanying drawings. Like reference numerals designate like elements throughout the specification, and overlapping descriptions of the elements will not be provided.

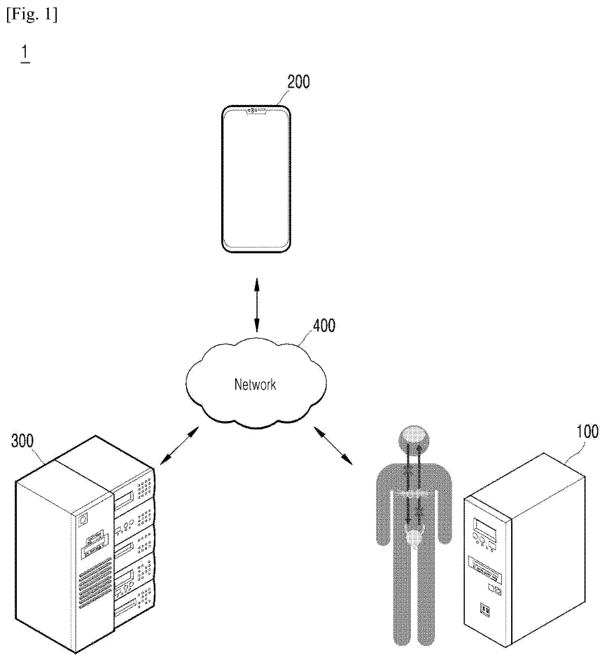

FIG. 1 schematically shows a bladder sensor tracking system environment for performing bladder monitoring based on a multi-channel strain bladder sensor according to an embodiment.

Referring to FIG. 1, a bladder sensor tracking system 1 according to an embodiment may perform the verification of a multi-channel strain bladder sensor (hereinafter, bladder sensor) and monitoring of the bladder, to which the bladder sensor is attached. That is, the bladder sensor tracking system 1 may perform the verification of the bladder sensor while observing the condition change of a measurement object after the attachment of the bladder sensor serving as a bladder to the bladder, before the bladder sensor is attached to the actual bladder to monitor the bladder condition.

In an embodiment, the bladder sensor is a highly flexible and stretchable strain sensor formed of a highly biocompatible material to monitor the bladder volume. Therefore, in one embodiment, the change in bladder volume can be appropriately monitored on the basis of the bladder sensor.

The bladder is a stretchable sac-like organ that stretches its length by up to 3 times when full of urine. Also, the muscle walls of the bladder contract for urination, becoming thicker and firm when empty.

The Young's modulus of the bladder is known to be 0.76 MPa for rat's bladder and 0.25-0.26 MPa pig's or human's bladder. Especially, the pig's bladder is known to have nearly the same characteristics as the human bladder, and thus is very suitable for research to treat patients with bladder dysfunction.

In other words, the bladder is a soft organ, known to have a low Young's modulus. The Young's modulus may also be referred to as elastic modulus, Young's coefficient, tensile or compressive elastic modulus, elastic modulus, or the like. It refers to an elastic modulus, indicating the degree to which an object is stretched and strained, and corresponds to a mechanical property for measurement of tensile or compressive stiffness of a solid material when a force is applied thereto in the length direction.

Therefore, the bladder sensor of one embodiment may be formed of a material that is highly biocompatible, has a low Young's modulus, and is sufficiently elastic (e.g., Ecoflex).

The bladder sensor according to one embodiment may be configured as a resistance change sensor for convenience of monitoring. To this end, the bladder sensor may be configured by forming a thin film of carbon nanotubes (CNTs), which is stretchable conductive material, on Ecoflex.

In one embodiment, gold-carbon nanotube (AuCNT) composites may be formed on the CNT thin film to compensate for low sensitivity occurring when only CNTs are used.

In one embodiment, a bladder sensor with multiple channels (e.g., three) may be used to track the movement of the bladder wall shown due to the expansion of the bladder. Hereinafter, a stick-shaped bladder sensor will be descried as an embodiment, but this is for convenience of explanation since a multi-channel bladder sensor is also fabricated through the same process as the stick-shaped bladder sensor.

In one embodiment, the bladder sensor can be verified by tracking the resistance change depending on the flow rate to measure the performance. In one embodiment, for the treatment of bladder diseases through neuro-stimulation, an implantable bladder sensor that has been verified may be used to monitor the bladder condition on the basis of changes in bladder volume.

TABLE 1

| Materials | Young's modulus (kPa) | Stretchability (%) |
|---|---|---|
| Rat bladder | 760 | — |
| Pig bladder | 260 | 200 |
| Human bladder | 250 | — |
| PDMS | 1840 | 133 |
| Eocflex 0050 | 83 | 980* |
| Eocflex 0030 | 69 | 900* |
| TPU | 3340 | 331 |

*by Smooth-On, Inc

For the fabrication of a bladder sensor, which is a resistive strain sensor for bladder monitoring, it is important to select materials that match the characteristics of the bladder mentioned above.

Table 1 compares Young's modulus and the stretchability among materials used in conventional bladder sensors and bladders. When two materials with different Young's modulus are attached in parallel, total Young's modulus is the sum of modulus of these two materials. Therefore, materials with low Young's modulus need to be used to make the bladder encounter less stress as the bladder expands. In addition, materials with high stretchability (at least 200% strain) need to be used to stretch with the bladder wall as it expands.

As shown in Table 1, among the materials that are mainly used in flexible and stretchable biological electrodes, polydimethylsiloxane or dimethylpolysiloxane (PDMS) has too low stretchability to be applied to bladder. Although thermoplastic polyurethane (TPU) has more than 300% strain stretchability, its Young's modulus is too high compared with the bladder.

Therefore, Ecoflex is appropriate as a substrate material for a bladder sensor, which has more than 900% strain stretchability and a Young's modulus of one-third that of pig's bladder.

Additionally, a stretchable conductive material is also required to stably maintain electrical conductivity of the bladder sensor when the bladder sensor is stretched. Many conductive materials are used in bladder sensors. Among them, CNT is capable of up to 500% strain when used along with Ecoflex.

Meanwhile, there are various options for the fabrication of CNT thin films, including spray coating, spin coating, chemical vapor deposition (CVD) growth, stamping, and inkjet printing. Among them, simple and low-cost spray coating may be applied, but there are not limitations on other options.

In one embodiment, flexible and stretchable Ecoflex and CNT may be used in the bladder sensor to measure the volume of the bladder. In addition, an AuCNT composite may be further coated on the CNT thin film to enhance the performance of the bladder sensor.

Hereinafter, a fabrication process of the bladder sensor will be described in more detail by using a stick-shaped bladder sensor as an example.

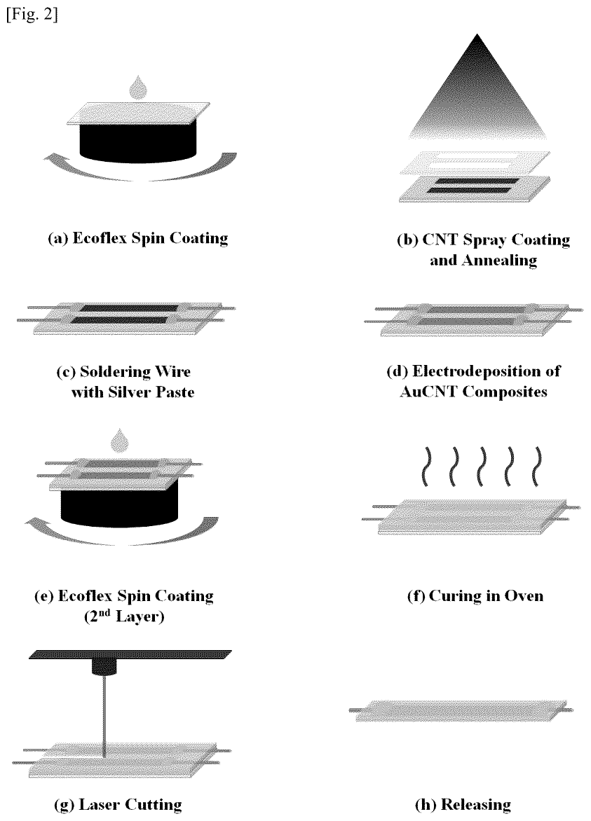

FIG. 2 shows a fabrication process of a bladder sensor according to an embodiment.

As shown in FIG. 2, the fabrication process of the bladder sensor begins from spin coating of a photoresist (e.g., AZ9260; AZ Electronic Materials Ltd.), to be used as a sacrificial layer, at 2,400 rpm/60 s (10 μm) on a 2-mm thick glass plate.

Then, in an embodiment, Ecoflex (e.g., Ecoflex 00-50; Smooth-On Inc.) as a substrate material was spin coated on the photoresist layer. Parts A and B of Ecoflex were mixed at 1:1 and poured on the photoresist layer (see FIG. 2(a)).

In an embodiment, air bubbles in Ecoflex are removed using a vacuum pump, and then the Ecoflex layer is spin coated at 1,000 rpm/60 s (70 μm). Particularly, if the time taken from mixing Parts A and B to spin coating is more than 10 minutes, Ecoflex may be cured to affect the spin coating of the thin Ecoflex layer. In an embodiment, the spin-coated Ecoflex layer is cured at room temperature for 30 minutes.

Next, in an embodiment, a polyimide film mask (25 μm) patterned by laser cutting is placed on the cured Ecoflex layer, and the resultant structure is placed on a hot plate at 85° C. for CNT spray coating.

For spray coating of a CNT thin film, a CNT spray solution needs to be prepared in advance. In an embodiment, 3 mL of a commercial 3 wt % multi-wall carbon nanotube (MWCNT) water dispersion (US Research Nanomaterials Inc., outer diameter: 20-30 nm, and length: 10-30 μm) was diluted in 110 ml of 0.1 wt % isopropyl alcohol (IPA). In an embodiment, the mixture may be sonicated for 1 hour at room temperature, and during this process, the temperature should not exceed 35° C.

Next, in an embodiment, the CNT spray solution is sprayed on the Ecoflex layer and the polyimide mask on the 85° C. hot plate (spray pressure: 2.4 bar, flow rate: 10 mL/min), as shown in FIG. 2(b). After spraying, the polyimide mask was removed from the Ecoflex layer, and the sensor was cleaned with IPA. Particularly, the adhesion of the Ecoflex layer and CNT thin film is still weak, so care needs to be taken to prevent the CNT thin film from peeling off. In an embodiment, the cleaned sensor is placed on a hot plate for 10 min, and then annealed in a convection oven at 150° C. for 30 min. This step is for enhancing the adhesion of the Ecoflex layer and the CNT thin film.

In an embodiment, copper wires with a length of 4 cm are bonded on the exposed CNT thin film by using a silver paste (see FIG. 2(c)), and the bonding portions are sealed with a silicone elastomer to thereby prevent the breakage of the silver paste during stretching.

Next, in an embodiment, an AuCNT composite is deposited on the CNT thin film by using an electrodeposition method (by which a material is formed on an electrode by electrolysis). For this method, an electrodeposition solution needs to be prepared. A short MWCNT powder (US Research Nano-materials Inc., outer diameter: <7 nm, length: 0.5-2 μm) was dispersed in a commercial gold plating solution (TSG-250, Transene company Inc.) at 1 mg/mL, and the solution was sonicated for 1 hour.

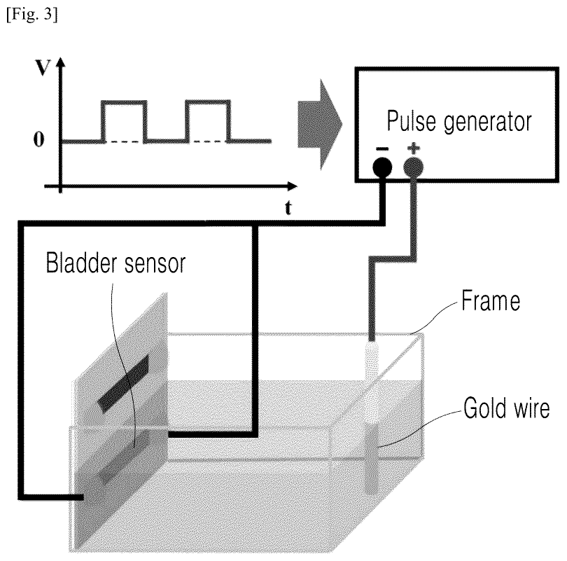

FIG. 3 shows the electrodeposition of an AuCNT composite according to an embodiment.

Referring to FIG. 3, in an embodiment, a gold wire and the bladder sensor may be connected to an anode and a cathode of a pulse generator, respectively, and the device may be inserted into a 3D-printed frame. The inner width of the frame may be 5 cm, and the distance between the center of the bladder sensor and the gold wire may be about 4.8 cm.

In an embodiment, the electrodeposition solution is added into the frame, and then a monophasic pulse (1.5 V, 1 Hz, 50% duty cycle) is applied for 8 minutes. The silicone elastomer surrounding the silver paste can prevent the AuCNT composite from being deposited on the silver paste, which is relatively more conductive than the CNT thin film.

Next, in an embodiment, a second Ecoflex layer (70 μm) is spin coated (see FIG. 2(e)), cured in a convection oven at 70° C. for 2 hours (see FIG. 2(f)), and then patterned into the shape of the bladder sensor by a laser cutting system (see FIG. 2(g)). Last, the photoresist layer as a sacrificial layer is dissolved in acetone to release the bladder sensor from the glass plate (see FIG. 2(h)).

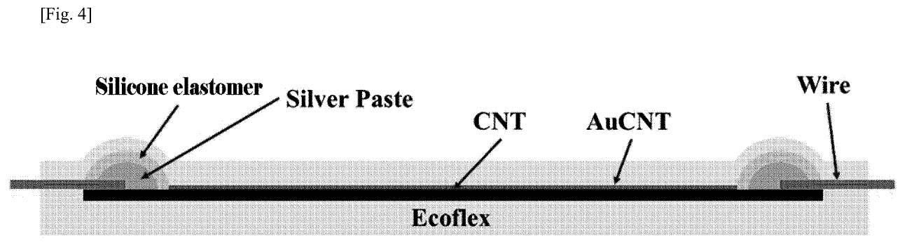

FIG. 4 shows a cross-sectional view of a bladder sensor according to an embodiment.

Referring to FIG. 4, a cross-sectional view of the AuCNT-containing bladder sensor fabricated through the above-described process may be shown. In an embodiment, a commercial polyimide film may be used for the AuCNT-containing bladder sensor. Polyimides (PIs) are polymer materials with high thermal stability, and exhibit excellent mechanical strength, chemical resistance, weather resistance, heat resistance, and insulation, as well as electrical properties such as a low dielectric constant, according to chemical stability of imide rings. Polyimides are attracting attention as lightweight and flexible high-functional polymer materials in the fields of displays, memories, solar cells, and the like.

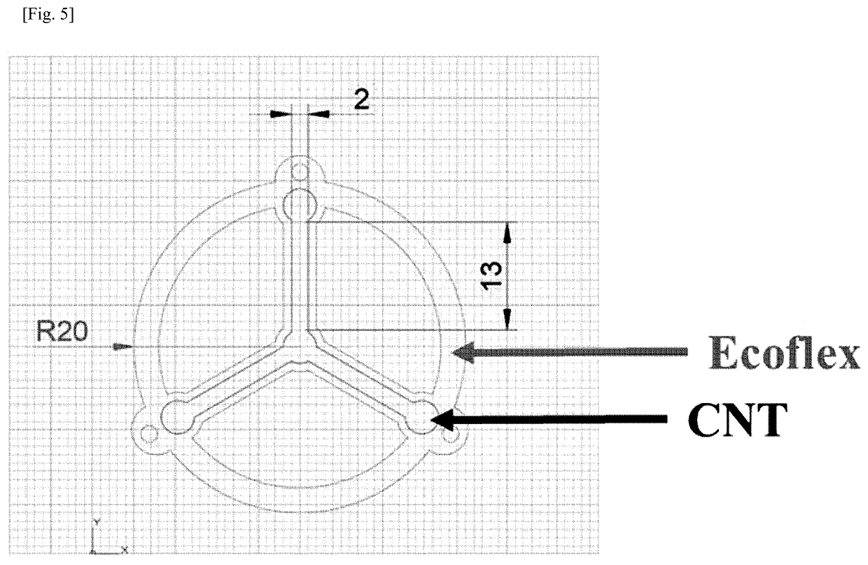

FIG. 5 shows a design of a multi-channel bladder sensor according to an embodiment.

As shown in FIG. 5, in an embodiment, a multi-channel bladder sensor capable of tracking the direction of expansion of the bladder wall can be fabricated by using the same process as the bladder sensor fabricating process, which has been described above using the stick-shaped bladder sensor as an example. This multi-channel bladder sensor may be verified by measuring resistance characteristics of each channel, wherein the resistance of each channel of the multiple channels may be measured simultaneously via parallel connections.

In this multi-channel bladder sensor, an AuCNT composite may be plated separately for each channel. Since the AuCNT bladder sensor is fabricated using electrodeposition, it is very difficult to control the randomly deposited composite as well as a gauge factor of each channel. Therefore, the expansion of each channel of the multi-channel bladder sensor is indirectly determined as follows.

$$\frac{Δ {R}_{n} / {R}_{0 , n}}{{GF}_{n}} \qquad [ Equation 1 ]$$

In the equation 1, n is the channel number.

In an embodiment, the relative expansion for each channel may be expressed as shown in Equation 2. In an embodiment, it is assumed that when the resistance is R₀, the relative expansion is 1.

$$\frac{Δ {R}_{n} / {R}_{0 , n}}{{GF}_{n}} + 1 \qquad [ Equation 2 ]$$

The gauge factor of each channel may be calculated by measuring the resistance at 0%, 100%, and 200% using the multimeter, after 20 times of stretching in up to 200% strain of each channel.

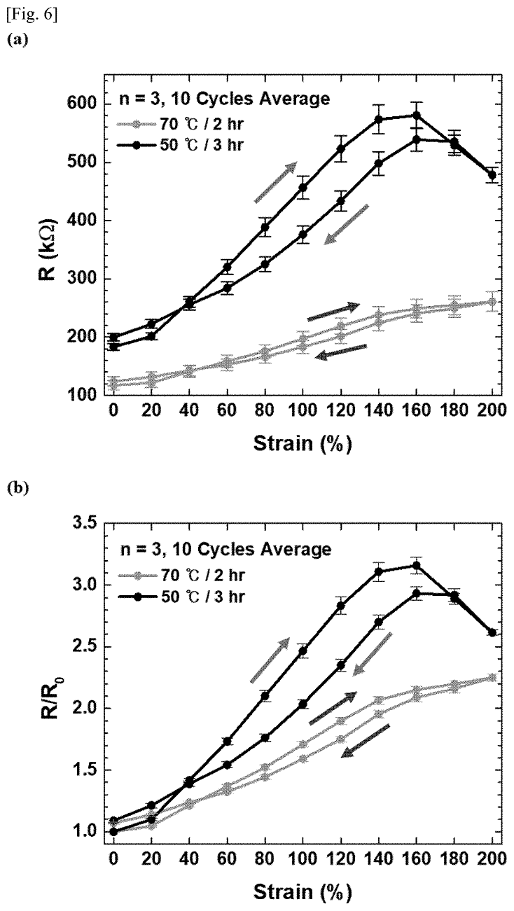

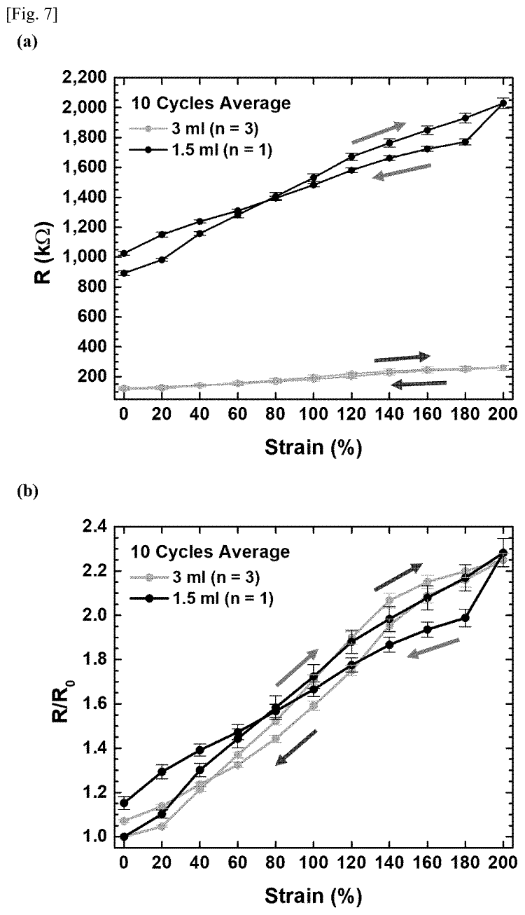

FIGS. 6 and 7 show the optimization of process conditions according to an embodiment.

In one embodiment, in order to identify the optimized process conditions for the fabrication of the bladder sensor, a test was conducted to compare the characteristics of the bladder sensor depending on the curing temperature and the spray amount for the second Ecoflex layer, which greatly affect the performance of the bladder sensor.

In one embodiment, a bladder sensor formed of Ecoflex-CNT without the addition of an AuCNT composite was tested.

As for the performance of the bladder sensor depending on the curing temperature of the second Ecoflex layer, the curing temperature of liquid Ecoflex may affect the formation of an Ecoflex-CNT nanocomposite.

In one embodiment, three types of bladder sensors were fabricated according to curing temperatures of 50° C., 70° C., and 85° C., respectively, and the fabrication conditions other than the curing temperature were as described in the fabrication process of the bladder sensor with reference to FIG. 2.

FIG. 6 compares the characteristics of CNT bladder sensors depending on the curing temperature between 50° C. and 70° C. FIG. 6(a) shows the resistance depending on the strain, and FIG. 6(b) shows the resistance change rate depending on the strain.

FIG. 6 shows the resistance characteristics of the CNT bladder sensors fabricated with curing temperatures of 50° C. and 70° C., within 200% strain rages.

The bladder sensors fabricated with a curing temperature of 50° C. showed a smallest resistance value of 183±30 kΩ (n=3) at 0% strain, and a largest value of 581±123 kΩ (n=3) at 160% strain. The bladder sensors fabricated with a curing temperature of 70° C. showed a smallest resistance value of 117±46 kΩ at 0% strain, and a largest value of 261±92 kΩ at 200% strain.

In addition, the maximum resistance change rates were 3.16±0.37 (n=3) and 2.25±0.16 (n=3) for curing temperatures of 50° C. and 70° C., respectively. The resistance change rate of the bladder sensors fabricated with a curing temperature of 50° C. was higher than that of the bladder sensors fabricated with a curing temperature of 70° C. However, the resistance change rate by a curing temperature of 50° C. had a maximum point at 160% strain. Therefore, such a curing temperature may not be suitable for the bladder sensors within 200% strain.

The bladder sensors fabricated with a curing temperature of 85° C. were unable to be measured for their resistance characteristics since the bladder sensors were opened even in a small strain. So, it can be determined that a curing temperature of 85° C. is also not suitable for the bladder sensors within 200% strain, and a curing temperature of 70° C. is the most suitable condition for fabrication of bladder sensors.

FIG. 7 compares the resistance characteristics of CNT bladder sensors according to the CNT dispersion. FIG. 7(a) shows the resistance depending on the strain, and FIG. 7(b) shows the resistance change rate depending on the strain.

In one embodiment, as for the performance of the bladder sensors according to the amount of CNT sprayed, the characteristics of the CNT bladder sensors may be compared according to the amount of MWCNT water dispersion where 1.5 mL and 3 mL of CNT solutions were used, respectively. Particularly, the densities of the CNT spray solutions were the same at 0.1 wt %, and thus the amount of CNT spray solution for the latter was twice as large as that of the former.

Referring to FIG. 7, the bladder sensors fabricated under 1.5 mL of the MWCNT water dispersion showed a smallest resistance value of 893±49 kΩ (n=1 and 10 cycles average) and a largest value of 2030±107 kΩ (n=1 and 10 cycles average) at 200% strain.

Also, the bladder sensors fabricated under 1.5 mL of the MWCNT water dispersion showed a smallest resistance value of 117±46 kΩ (n=3) and a largest value of 261±92 kΩ (n=3) at 200% strain. In addition, the maximum resistance change rates were 2.28±0.20 (n=1 and 10 cycles average) and 2.25±0.16 (n=3) for 1.5 mL and 3 mL of the MWCNT water dispersions, respectively, indicating very similar values.

However, the resistance value in the 1.5 mL case was about 7 times higher than that in the 3 mL case, and these values were difficult to distinguish when the network of the bladder sensor was broken. Therefore, in an embodiment, it can be determined that 3 mL of a MWCNT water dispersion is a most suitable condition for fabrication of the sensors.

Meanwhile, in an embodiment, an AuCNT composite may be additionally deposited on the CNT film using electrodeposition, in order to increase the sensitivity of Ecoflex-CNT bladder sensors.

Particularly, as for fabrication conditions other than the additional deposition of the AuCNT composite, it can be determined that a curing temperature of 70° C. within 200% strain corresponds to most suitable conditions in the fabrication of bladder sensors and 3 mL of an MWCNT water dispersion corresponds to most suitable conditions in the fabrication of bladder sensors.

Hereinafter, a comparison was made between the resistance characteristics of bladder sensors composed of CNTs and the resistance characteristics of bladder sensors composed of an additionally deposited AuCNT composite.

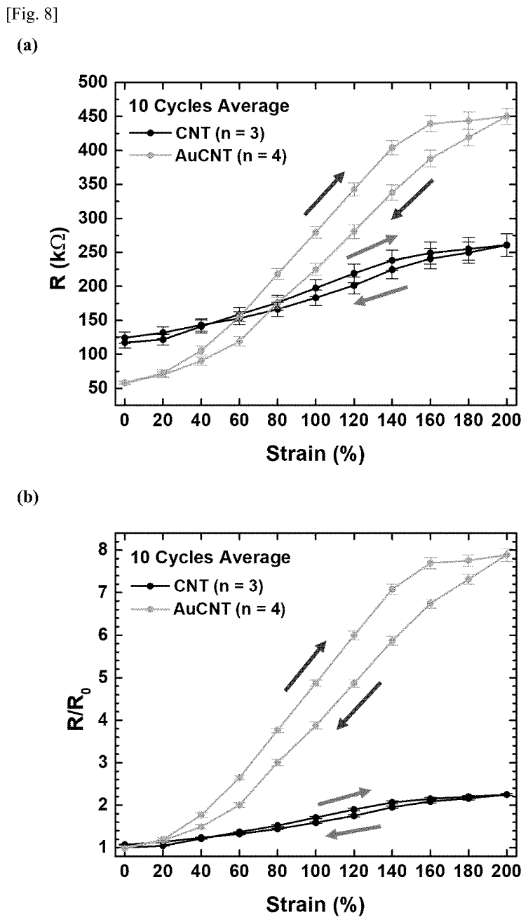

FIG. 8 compares CNT bladder sensors and AuCNT-added bladder sensors according to an embodiment.

In other words, FIG. 8 shows the results of measuring the resistance characteristics of two types of bladder sensors, within the strain range of 200%. FIG. 8(a) shows the resistance depending on to the strain, and FIG. 8(b) shows the resistance change rate depending on the strain.

Referring to FIG. 8, the bladder sensors composed of CNTs showed a resistance value of 117±46 kΩ (n=3) at 0% strain and a resistance value of 261±92 kΩ (n=3) at 200% strain. The bladder sensors composed of the AuCNT composite showed a resistance value of 58±14 kΩ (n=4) at 0% strain and 450±77 kΩ (n=4) at 200% strain. These bladder sensors also showed maximum resistance change rates of 2.25±0.16 (n=3) and 7.89±0.93 (n=4) at 200% strain, respectively.

In addition, the gauge factors of these bladder sensors were calculated as 0.625±0.073 (n=3) and 3.455±0.463 (n=4), respectively. These results indicate that the gauge factor of the bladder sensors composed of an AuCNT composite was improved by about 5 times than that of the bladder sensors composed of CNTs, showing that the initial resistance was 2 times lower. As a result, in an embodiment, the deposition of the AuCNT composite improved the sensitivity of the Ecoflex-CNT bladder sensors within the strain range of 200%.

Meanwhile, in an embodiment, the resistance depending on the strain may be measured to evaluate the performance of stick-shaped bladder sensors.

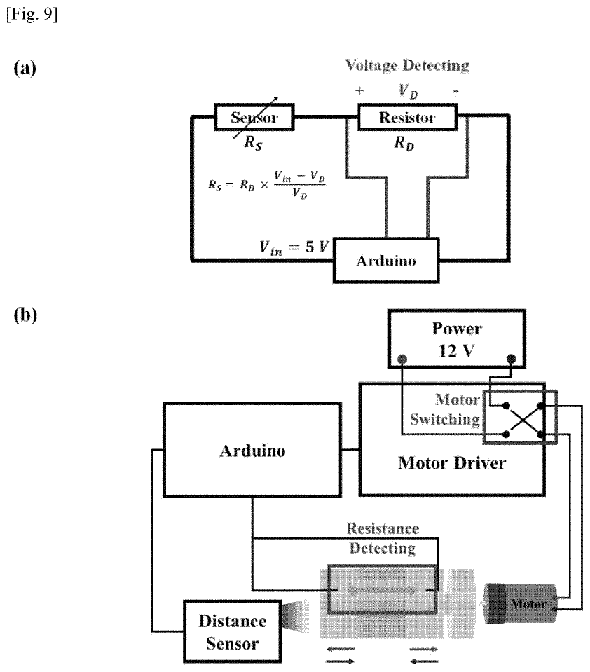

FIG. 9 illustrates a strain resistance measurement method by a stick-shaped bladder sensor according to an embodiment.

FIG. 9(a) illustrates the resistance detection by the voltage division rule, and FIG. 9(b) schematically shows a device for measuring the strain resistance of a stick-shaped bladder sensor.

The device for measuring the strain resistance of the stick-shaped bladder sensor shown in FIG. 9(b) (hereinafter, a resistance measurement device) may be configured to include a control board (e.g., Arduino), a motor, and a distance sensor. The control board may mean an open source computing platform and a software development environment based on a microcontroller board.

The resistance measurement device according to one embodiment may employ the voltage division rule to measure the resistance of the stick-shaped bladder sensor (see FIG. 9(a)).

A detecting resistor used in one embodiment may be a 100-kΩ resistor, and may calibrate the difference between the ideal value of the detection voltage and the actual input signal of the control board. In one embodiment, the two values may be matched by measuring the actual input voltage of the control board through 10-kΩ, 100-kΩ, and 1-MΩ resistors.

In one embodiment, considering the noise of the input signal of the control board, the average resistance may be determined by reading 200 signals at one time.

In one embodiment, a motor may be used to extend and contract the stick-shaped bladder sensor. Particularly, the length of the stick-shaped bladder sensor may be detected through an infrared distance sensor (GP2Y0A41SK0F). In one embodiment, a motor drive (L298N) for switching the extending and contracting of the stick-shaped bladder sensor may be used to switch the direction of rotation of the motor when the stick-shaped bladder sensor reaches a desired length. Additionally, the measured data was stored on the SD card in a text file format by the SD card module.

In one embodiment, the resistance measurement device may be used to repeatedly measure the resistance changes within 200% strain for 40 cycles. The strain change rate was 200% strain/min, and the resistance was measured at every 20% strain step. The performance of each stick-shaped bladder sensor may be specified as an average of later 10 cycles.

Meanwhile, one embodiment relates to a multi-channel bladder sensor to be applied to bladder monitoring, and thus it is preferable to measure the resistance change of the multi-channel bladder sensor depending on the volume by using an object that can replace the bladder, such as a balloon, or an animal bladder.

In one embodiment, the bladder sensor can be verified by measuring the resistance characteristics depending on the volume by using a processor, a circulator (pump) for circulating a liquid, a flow sensor, and the like.

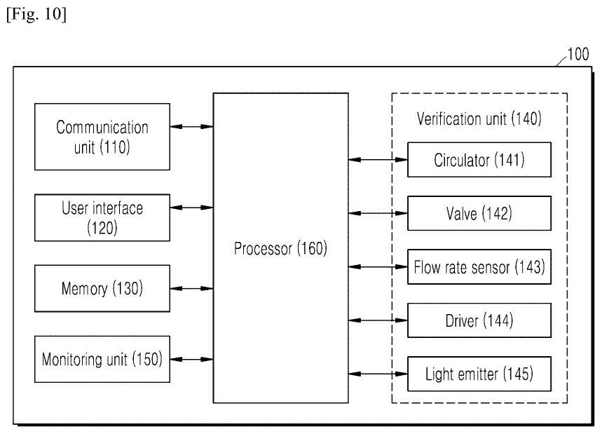

FIG. 10 is a block diagram schematically showing a bladder sensor tracking system for performing the verification of a multi-channel strain bladder sensor and the monitoring thereby according to an embodiment.

Referring to FIG. 10, a bladder sensor tracking system 100 may include a communication unit 110, a user interface 120, a memory 130, a verification unit 140, a monitoring unit 150, and a processor 160, whereby the verification of the bladder sensor and the bladder sensor-based monitoring for the bladder condition may be performed.

In one embodiment, the bladder sensor tracking system 100 may include: a multi-channel strain bladder sensor verification device for performing the verification of the bladder sensor; and a bladder monitoring device based on the multi-channel strain bladder sensor for performing the bladder sensor-based monitoring of the bladder condition, wherein each of the devices may be performed separately by each processor or may be performed by the same processor. Hereinafter, the performing of each of the devices by the same processor is described. According to embodiments, each of the devices may be configured separately from the bladder sensor tracking system 100.

The communication unit 110 may provide a communication interface necessary to interwork with the network 300 to provide transmission/reception signals in the form of packet data between external devices. The communication unit 110 may include hardware or software necessary to transmit and receive signals, such as control signals or data signals, via wired/wireless connection with other network devices.

That is, the processor 160 may receive various types of data or information from an external device connected through the communication unit 110 and may also transmit various types of data or information to the external device.

In one embodiment, the user interface 120 may include an input interface for inputting, therethrough, user requests and commands for controlling the operations (e.g., changing the flow rate sensing value to adjust the amount of liquid flowing into and out of a measurement object, changing the setting of circulators and valves, changing the standard range for monitoring the bladder condition, changing the criteria for determining incomplete urination, and the like) of the bladder sensor tracking system 100.

In one embodiment, the user interface 120 may include an output interface for outputting, therethrough, multi-channel strain bladder sensor verification results and bladder condition monitoring results based on the multi-channel strain bladder sensor. That is, the user interface 120 may output the results according to user requests and commands. The input interface and output interface of the user interface 120 may be implemented in the same interface.

The memory 130 stores various types of information necessary for controlling (computing) the operations of the bladder sensor tracking system 100 and stores control software, and may include a volatile or non-volatile recording medium.

The memory 130 may be connected to at least one processor 160 by an electrical or internal communication interface. When executed by processor 160, the memory 130 may store codes causing the processor 160 to control the bladder sensor tracking system 100.

Particularly, the memory 130 may include non-transitory storage media, such as magnetic storage media or flash storage media, or a transitory storage media such as RAM, but the scope of the present disclosure is not limited thereto. The memory 130 may include an internal memory and/or an external memory. The memory 130 may include volatile memory (such as DRAM, SRAM, or SDRAM), non-volatile memory (such as one-time programmable ROM (OTPROM), PROM, EPROM, EEPROM, mask ROM, flash ROM, NAND flash memory, or NOR flash memory), a storage device, for example, HDD or a flash drive (such as SSD, a compact flash (CF) card, an SD card, a micro-SD card, a mini-SD card, an Xd card, or a memory stick).

Additionally, the memory 130 may store information associated with an algorithm for performing learning according to the present disclosure. Additionally, various types of information required within a range for achieving the purpose of the present disclosure may be stored in the memory 130, and the information stored in the memory 130 may be updated by receiving data from a server or an external device or inputting data by a user.

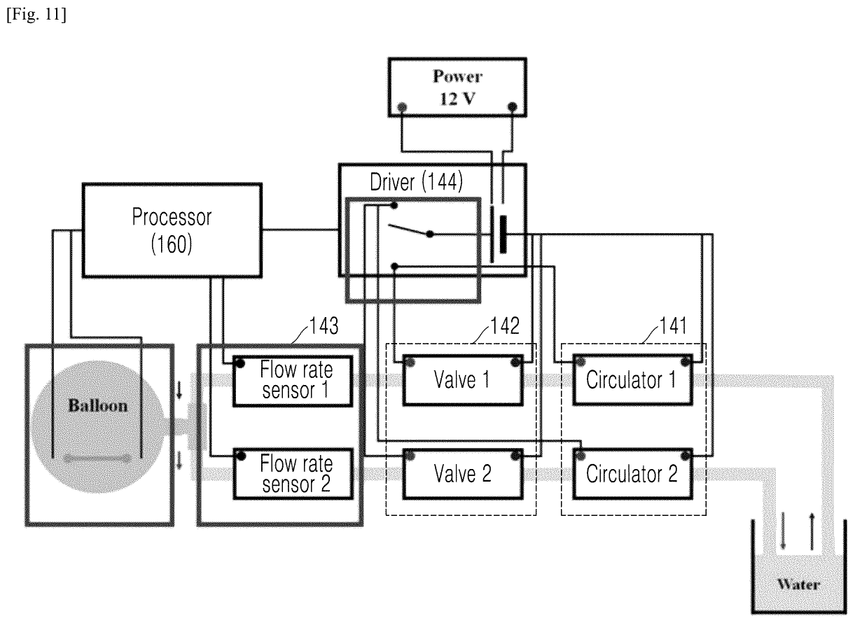

FIG. 11 schematically shows a verification unit for performing the bladder sensor verification according to an embodiment.

As shown in FIG. 11, the verification unit 140 may include: a pair of inlet and outlet lines connected to allow a liquid to be injected into a measurement object; a circulator 141 allowing a liquid to circulate; a valve 142 controlled to open and close; a flow rate sensor 143 for detecting the flow rates of the inlet and outlet lines; and a driver 144 for driving the circulator 141 and the valve 142. Particularly, the measurement object may include objects, such as a balloon, capable of replacing an actual bladder.

For example, the verification unit 140 requires both an inlet line and an outlet line, and thus two circulators 141, two valves 142, and two flow rate sensors 143. The circulator 141 may be a water pump (HS-WATER PUMP IV), and the valve 142 may be two solenoid valves (HDW-2120). In an embodiment, two lines (inlet and outlet lines) may be connected using a T-shaped tube.

In one embodiment, in the verification unit 140, the two lines were connected in parallel to output terminals of the driver 144. The driver 144 may be a motor driver (L298N). When the amount of water measured by the flow rate sensor 143 reached a desired level, the processor 160 may transmit a signal to the driver 144 to change the inflow and outflow of water. According to embodiments, a processor may be configured separately from the verification unit 140.

That is, in an embodiment, the inflow and outflow of a liquid with respect to the measurement object may be controlled through the verification unit 140.

The monitoring unit 150 provides the monitoring results on the bladder condition to a user, and may be configured as an interface for informing the bladder condition, that is, the timing of urination or the incomplete urination. According to embodiments, the monitoring unit may be implemented in the same configuration as the user interface 120.

The processor 160 may control the overall operations of the bladder sensor tracking system 100. Specifically, the processor 160 is connected to the bladder sensor tracking system 100 including the memory 130, and can control the overall operations of the bladder sensor tracking system 100 by executing at least one command stored in the memory 130.

The processor 160 may be implemented in various manners. For example, the processor 160 may be implemented as at least one of an application specific integrated circuit (ASIC), an embedded processor, a microprocessor, hardware control logic, a hardware finite state machine (FSM), and a digital signal processor (DSP).

The processor 160 is a type of central processing unit, and can control the operations of the bladder sensor tracking system 100 by running the control software mounted on the memory 130. The processor 160 may include any type of device capable of processing data. The term “processor” may refer to a data processing device built into the hardware, which as a physically structured circuit in order to perform functions expressed by codes or instructions included in a program.

In one embodiment, the processor 160 may perform the verification of the bladder sensor and monitor the bladder condition on the basis of the verified bladder sensor.

First, a multi-channel strain bladder sensor verification method for verifying the bladder sensor is described.

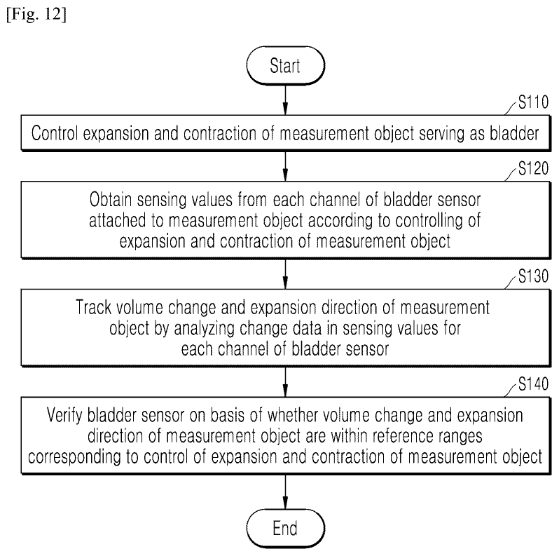

FIG. 12 is a flow chart for illustrating a multi-channel strain bladder sensor verification method according to an embodiment.

As shown in FIG. 12, the processor 160 controls the expansion and contraction of a measurement object serving as a bladder in step S110, and acquires sensing values from each channel of a bladder sensor attached to the measurement object according to the control of expansion and contraction of the measurement object in step S120.

The processor 160 controls the inflow and outflow of a liquid with respect to the measurement object through a pair of inlet and outlet lines connected so as to inject the liquid into the measurement object, wherein the processor drives the circulator 141 and the valve 142 through the driver 144 on the basis of the flow rate sensing values from the flow rate sensor 143, thereby controlling the inflow and outflow of the liquid with respect to the measurement object. The flow rate of water may be set to be 10 mL/s.

In step S130, the processor 160 analyzes change data in sensing values for each channel of the bladder sensor to track the volume change and expansion direction of the measurement object.

In such a case, the processor 160 measures the extended length of a base substrate of the bladder sensor on the basis of the resistance values of each channel of the bladder sensor, and calculates the length of expansion in the axial direction corresponding to each channel according to the extended length of each channel.

Such a bladder sensor may be a resistive sensor in which as the measurement object expands, the conductivity between coated electrical conductors decreases, resulting in an increase in resistance value.

In step S140, the processor 160 verifies the bladder sensor on the basis of whether the volume change and expansion direction of the measurement object are within reference ranges corresponding to the control of the expansion and contraction of the measurement object.

Particularly, the processor 160 injects a predetermined amount of a liquid into the measurement object in an initial state, which is expanded corresponding to the reference bladder volume, thereby checking whether the length of expansion in each expansion direction of the bladder sensor is within the expansion range corresponding to the amount of the liquid injected.

In addition, the processor 160 allows the injected liquid to flow out to check whether the length of expansion in each expansion direction of the bladder sensor is within the normal range based on the length of expansion at the initial state.

For example, it may be checked whether the value of the detection voltage is within the reference normal range by using a detecting resistor of 100 kΩ along with 10-kΩ, 100-kΩ, and 1-MΩ resistors, and the difference between the ideal value of the detection voltage and the actual input signal of the verification unit 140 may be calibrated.

The bladder sensor of one embodiment may be configured as a structure where a carbon nanotube (CNT) film and an AuCNT composite are deposited on an Ecoflex base substrate as described above, and may be configured to have a three-channel sensor with channels spaced at intervals of 120 degrees. In addition, the bladder sensor may be configured to set a gauge factor indicating a resistance change in response to the strain according to the strain characteristics of the bladder.

In one embodiment, the verification of such a bladder sensor may be performed. Hereinafter, an embodiment of the verification of a multi-channel strain bladder sensor will be described.

In one embodiment, the resistance according to volume may be measured by applying the bladder sensor to a balloon-model with a diameter of 5 cm before applying the sensor to the bladder. Particularly, the bladder sensor was fixed on the balloon using a double-sided tape. In one embodiment, the tape may be fixed to the bottom of the silicone elastomer surrounding a silver paste.

In one embodiment, the resistance characteristics may be measured within a volume of 200 mL. The resistance may be measured every 10 mL of a volume, and the measurement may be repeated 10 cycles. Then, an average of later 5 cycles may be determined as the performance of the bladder sensor.

In one embodiment, the verification unit 140 may further include a light emitter 145. In one embodiment, for example, when a balloon is used as a measurement object, the state of the volume according to the volume of the balloon, that is, the resistance characteristic results, may be displayed using three LEDs. That is, green, yellow, and red LEDs may be sequentially turned on, when the volume of the balloon increase as 50 mL, 100 mL, and 150 mL.

In an embodiment, when the verification unit 140 first performs the verification, the resistance value of the bladder sensor is set to R₀, and the measurement of resistance was then repeated to calculate R/R₀, which determines whether the LED has been turned on or off.

In another embodiment, the bladder sensor may be applied to the extracted pig's bladder to allow for verification in a more realistic manner. The sensor may be fixed to the bladder wall using surgical suture (SK434, Black silk 4-0, AILEE CO., LTD). In an embodiment, the Ecoflex part of the bladder sensor around the wire bonding section may be sutured with the bladder wall, and a silicone elastomer, such as the one surrounded around a silver paste, may be used to prevent the sutured part from tearing when the bladder sensor is stretched.

In an embodiment, the resistance depending on the volume during the expansion of the bladder may be measured, and for example, 600 mL of water may be injected into the bladder and the resistance of the sensor may be measured by every 20 mL. The length of the bladder sensor at the maximum volume of the bladder may be measured to determine the stretched length of the bladder wall. However, the extracted bladder cannot contract the muscles of the bladder wall, and thus it is impossible that the bladder returns to its original size. Therefore, in an embodiment, one measurement may be attempted for one bladder.

Hereinafter, the results of experiments performed according to the embodiments of the above verification method will be described.

First, the results of experiments using a stick-shaped bladder are described.

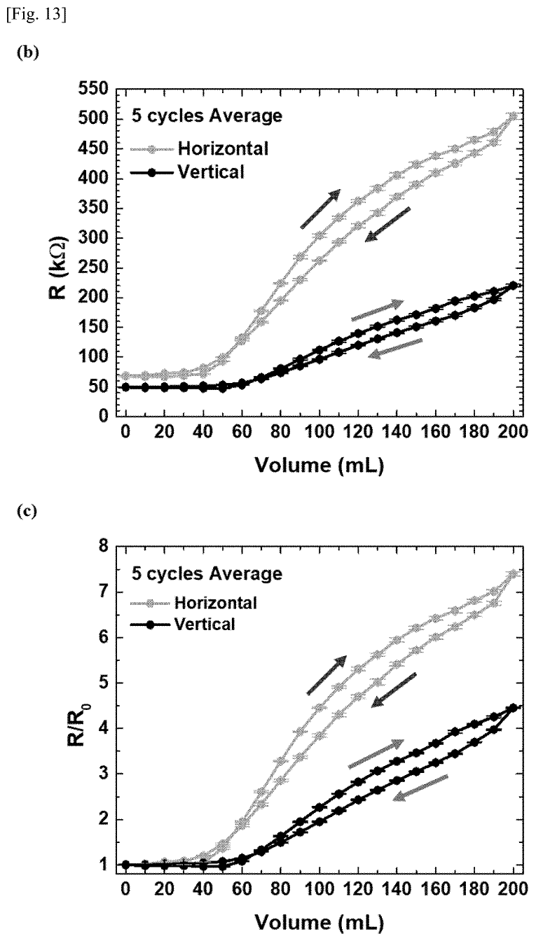

FIG. 13 illustrates the characteristics of a stick-shaped bladder sensor depending on the volume of a balloon model according to an embodiment.

In an embodiment, a bladder sensor composed of an AuCNT composite is applied to a balloon model to verify the performance of volume monitoring before the application to pig's bladders.

The same stick-shaped bladder sensors were attached to the same balloons in two directions, horizontally and vertically, respectively, to measure the resistance characteristics depending on the volume. At 200 mL of the volume, the lengths of the stick-shaped bladder sensors were 38 mm (90% strain) horizontally and 32 mm (60% strain) vertically. These results confirmed that the balloons used were stretched more in the horizontal direction.

FIGS. 13(a) and 13(b) show the results of measuring the resistance characteristics depending on the volume of the balloon by the stick-shaped bladder sensor. Referring to FIG. 13(b), the resistance change rate at 200 mL was 7.40±0.11 (5 cycles average) horizontally and 4.45±0.04 (5 cycles average) vertically. As a result of the repeated measurements, the standard deviation of the values measured in 5 cycles was within 2% of the average value. It can therefore be identified that the stick-shaped bladder sensor can measure the volume of the balloon repeatedly and stably.

Furthermore, the experimental results confirmed that as the balloon expanded, the balloon wall stretched more in the horizontal direction, like the results from the direct measurement of the stretched length of the stick-shaped bladder sensor. Resultantly, it can therefore be identified that the stick-shaped bladder sensor can measure the expansion of the balloon wall.

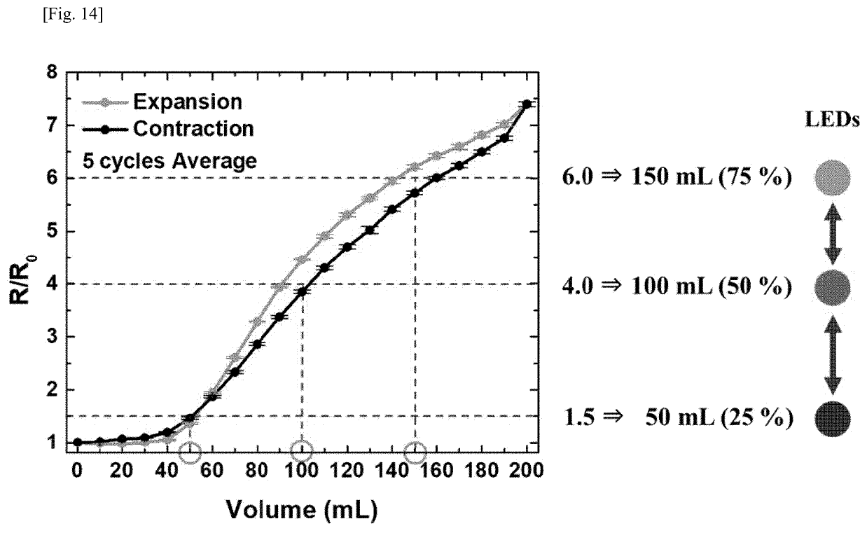

FIG. 14 illustrates a verification experiment using a light emitter according to an embodiment.

In an embodiment, an experiment of performing the verification of a stick-shaped bladder sensor through volume monitoring may be conducted by using a light emitter 145, on the basis of the results of the resistance characteristic depending on the volume measured as above (see FIG. 13).

The stick-shaped bladder sensor is relatively sensitive to volume changes, and thus the results resulting from the horizontal attachment of the bladder sensor may be used. FIG. 14 shows the parameters based on the results of measurement of resistance characteristics depending on the horizontal volume.

Three different colored LEDs were used. When the volume of the balloon was 50 mL, 100 mL, and 150 mL, the green, yellow, and red LEDs were turned on, and the resistance change rates of the stick-shaped bladder sensor were 1.5, 4.0, and 6.0, respectively.

When each LED was turned on, the volume of the balloon was calculated by using time and flow rates. As a result, there was an error of less than 1 second (10 mL) when each LED was turned on. Therefore, it can be identified through the experiment using the light emitter 145 that the volume of the balloon can be stably tracked.

Next, in an embodiment, an experiment on the verification of a stick-shaped bladder sensor was conducted by applying the stick-shaped bladder sensor to pig's bladder.

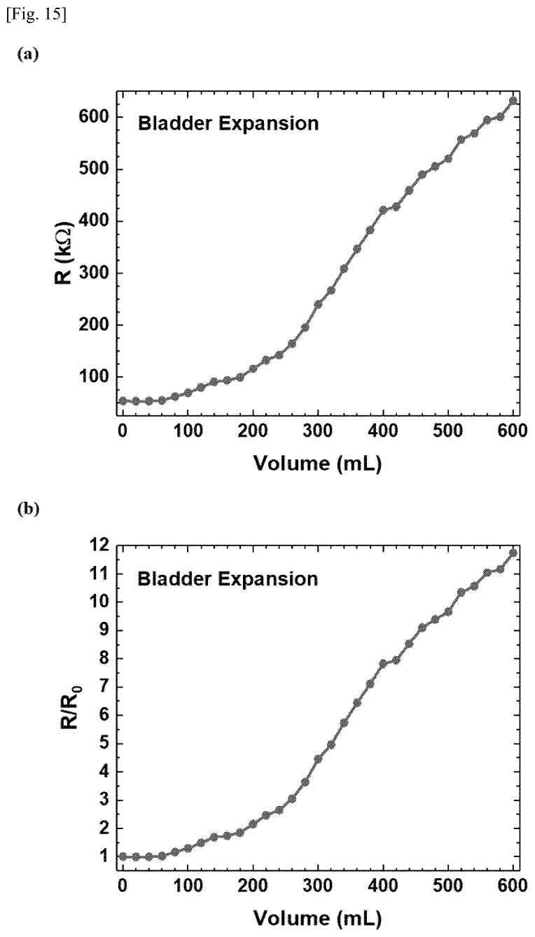

FIG. 15 illustrates a verification experiment of a stick-shaped bladder sensor by using pig's bladder according to an embodiment.

FIG. 15 is accosted with Ex-vivo verification experiments using pig's bladder. FIG. 15(a) shows the resistance depending on the volume, and FIG. 15(b) shows the resistance change rate depending on the volume.

A stick-shaped bladder sensor was attached to the wall of the extracted pig's bladder in order to measure the resistance characteristics depending on the volume. It was found that the stick-shaped bladder sensor attached to the bladder wall with surgical sutures and a silicone element was stable at a volume of 600 mL, known as the maximum volume of the pig's bladder. When the volume of the bladder was 600 mL, the stick-shaped bladder sensor was 50 mm long. Apart of the bladder wall, to which the stick-shaped bladder sensor was attached, showed 150% strain at the volume of 600 mL.

In one embodiment, the resistance characteristics of stick-shaped bladder sensor may be measured while the volume of the bladder expands to 600 mL. In an embodiment, the resistance may be measured by attaching the stick-shaped bladder sensor horizontally to the center of the bladder, and the results are shown in FIG. 15.

According to the graph in FIG. 15(b), when the volume of the bladder expanded to 600 mL, the resistance change rate increased to 11.75. It can therefore be identified that the stick-shaped bladder sensor can operate within the 600 mL volume range of the pig's bladder.

Meanwhile, the results in FIG. 15 were obtained using the pig's bladder and showed a higher resistance change rate than the results shown in FIG. 8, at the same strain of the stick-shaped bladder sensor composed of an AuCNT composite. For these results, it can be assumed that the pressure from the curvature of the bladder affected the resistance of the stick-shaped bladder sensor.

It can be confirmed through the experimental results of the above-described stick-shaped bladder sensor that a multi-channel bladder sensor (composed of AuCNT composite) fabricated on the basis of a stick-shaped bladder sensor can identify the volume of the bladder through the resistance change.

The balloon model has different degrees of expandability depending on the direction, and it was assumed through the above experiment that the pressure from the curvature of the bladder wall affects the resistance of the bladder sensor.

Therefore, in one embodiment, a multi-channel (3-channel) bladder sensor capable of tracking the expansion direction of the bladder as well as simply measuring the volume of the bladder may be used. In addition, the multi-channel bladder sensor may be less susceptible by the pressure from the curvature of the bladder wall since each channel of the multi-channel bladder sensor is shorter than the stick-shaped bladder sensor used in the above experiment.

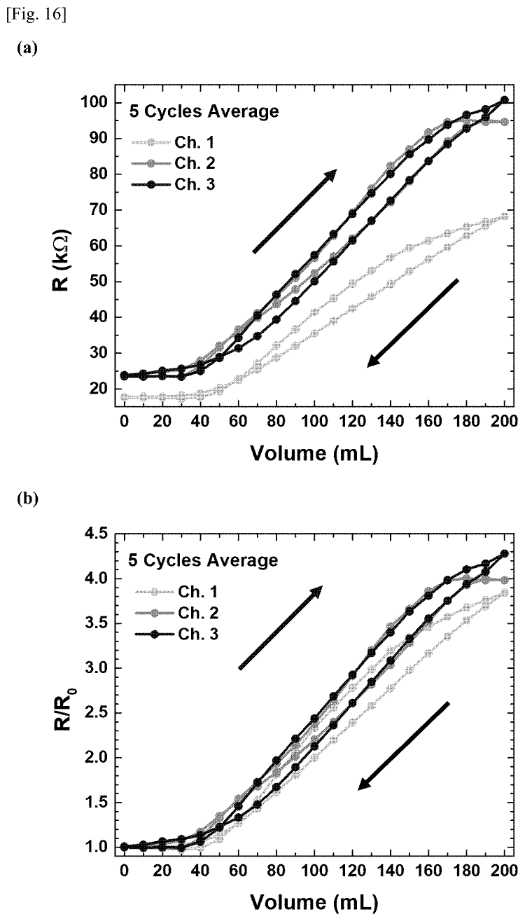

FIG. 16 illustrates the volume resistance characteristics of a multi-channel bladder sensor according to an embodiment.

FIG. 16 shows the result of applying a bladder sensor to a balloon model to measure the resistance characteristics depending on the volume. FIG. 16(a) shows the resistance depending on the volume, and FIG. 16(b) shows the resistance change rate depending on the volume.

In this experiment, the bladder sensor was attached to the center of the balloon with the channel 1 facing upwards. Also, this experiment was conducted in the range of 200 mL.

The graph in FIG. 16(b) shows the resistance change rate for each channel. The resistance change rate at the maximum volume of 200 mL for each channel was 3.84±0.10 (5 cycles average), 3.98±0.01 (5 cycles average), and 4.28±0.11 (5 cycles average), respectively.

However, in the bladder sensor, the AuCNT composite is deposited using electrodeposition, so the composition thereof is very random. Therefore, it is very difficult to control the gauge factor of each channel at the same level, and thus the length of the stretched bladder sensor cannot be measured using resistance change rates. Thus, the gauge factor of each channel was measured and applied to the resistance change rate.

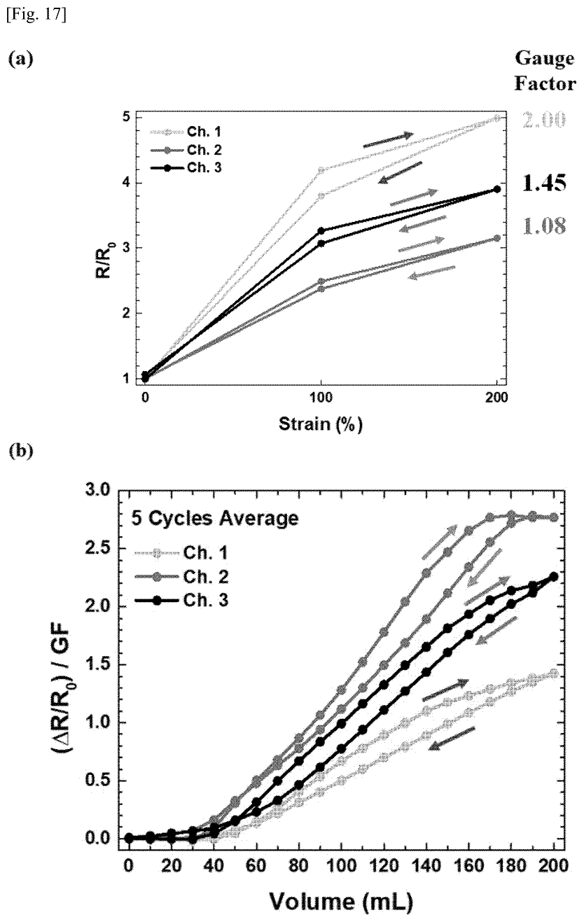

FIG. 17 shows the characteristics of the multi-channel bladder sensor used in the balloon model according to an embodiment.

FIG. 17(a) shows the gauge factor of each channel of the strain sensor used in the balloon model. The measured gauge factors of each channel were 2.00, 1.08, and 1.45. This gauge factor for each channel was applied to the resistance change rate of each channel to determine the relative expansion.

FIG. 17(b) shows the relative expansion of the bladder sensor employing each gauge factor. That is, the graph of FIG. 17(b) shows the relative expansion for each channel by using gauge factors, and the relative expansion values for each channel were 1.42, 2.77, and 2.26, respectively.

As shown in the graph of FIG. 17(b), the lengths of respective channels at 200 mL were 16 mm (60% strain), 25 mm (150% strain), and 22 mm (120% strain). Both two results showed a trend of channel 2>channel 3>channel 1. It can therefore be identified that the multi-channel bladder sensor can measure the relative expansion of the balloon in each direction.

Next, in one embodiment, the expansion direction of pig's bladder can be tracked using the bladder sensor, indicating that the relative expansion direction can be measured through resistance changes.

In one embodiment, the bladder sensor may be fixed on the wall of a pig's bladder by using surgical suture and a silicone elastomer, and the resistance depending on the volume may be measured using a multi-channel bladder sensor in order to track the relative expansion direction of the pig's bladder within 600 mL, as shown in the experiment about the stick-shaped bladder sensor.

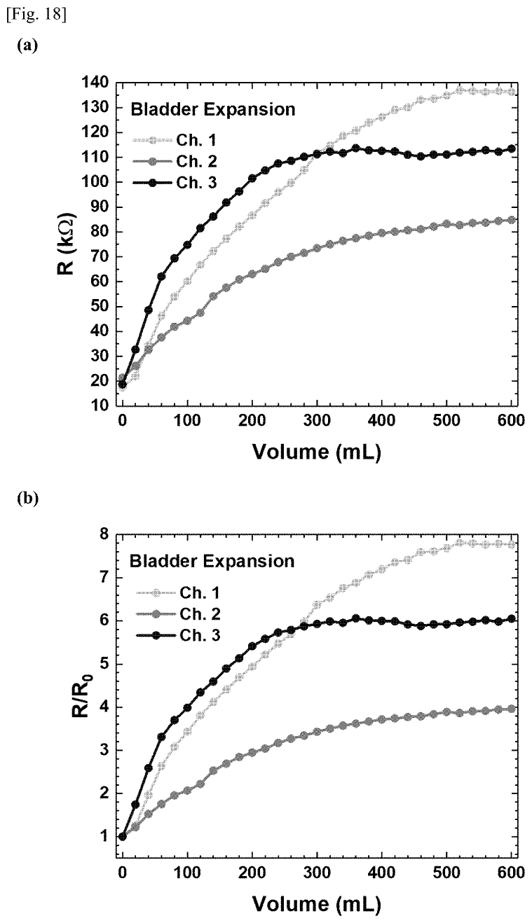

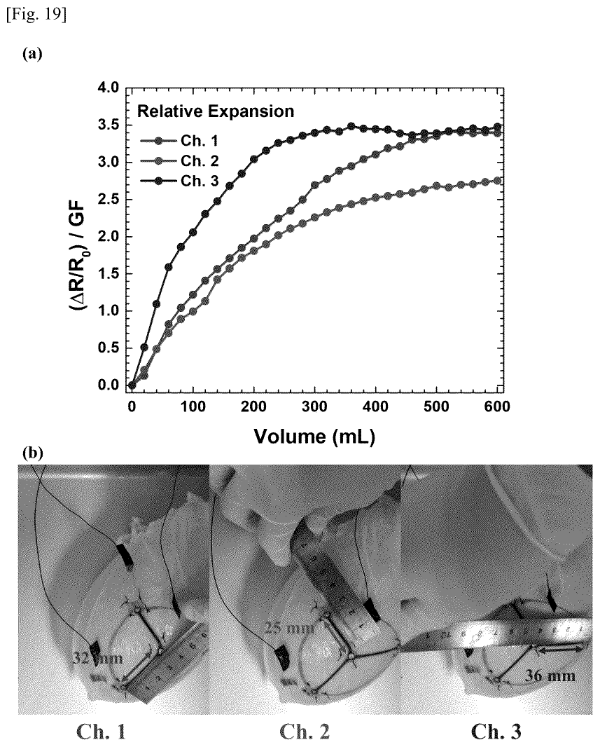

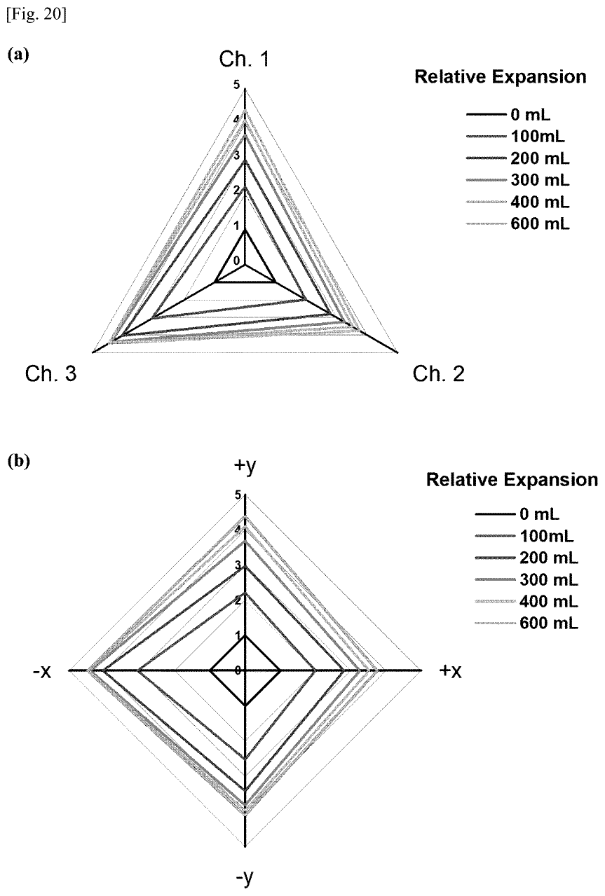

FIG. 18 illustrates the volume-resistance characteristics of the bladder sensor on the pig's bladder according to one embodiment. FIG. 19 shows the results of an experiment for detecting the expansion direction of the bladder sensor according to one embodiment, and FIG. 20 illustrates the bladder expansion depending on the volume according to one embodiment.

FIG. 18(a) shows the resistance depending on the volume, and FIG. 18(b) shows the resistance change rate depending on the volume. FIG. 19(a) shows the relative expansion of the bladder sensor employing each gauge factor, and FIG. 19(b) shows the length of each channel at a volume of 600 mL. FIG. 20(a) shows the expansion of the bladder depending on the volume for each channel, and FIG. 20(b) shows the expansion of the bladder depending on the volume on the x-y coordinates.

FIG. 18 shows the resistance change for each channel of the bladder sensor depending on the volume of the pig's bladder. According to FIG. 18(b), when the volume of the bladder was 600 mL, the resistance change rates of the respective channels were 7.77, 3.96, and 6.05, respectively.

FIG. 19 shows the relative expansion of the bladder at a volume of 600 mL and the length of each channel. In FIG. 19(b), when the volume of the bladder was 600 mL, the relative expansion values of the channels were 3.39, 2.75, and 3.48, respectively. As shown in FIG. 19(b), the lengths of the channels were 32 mm (220% strain), 25 mm (150% strain), and 36 mm (260% strain), respectively. That is, these results showed the same tendency as the results in the balloon-model.

From the result of FIG. 19(a), it can be indirectly identified which direction the bladder wall expanded more depending on the volume of the bladder. Referring to FIG. 20, it can be intuitively identified on the basis of the above results in which direction the bladder expanded as the bladder volume increased.

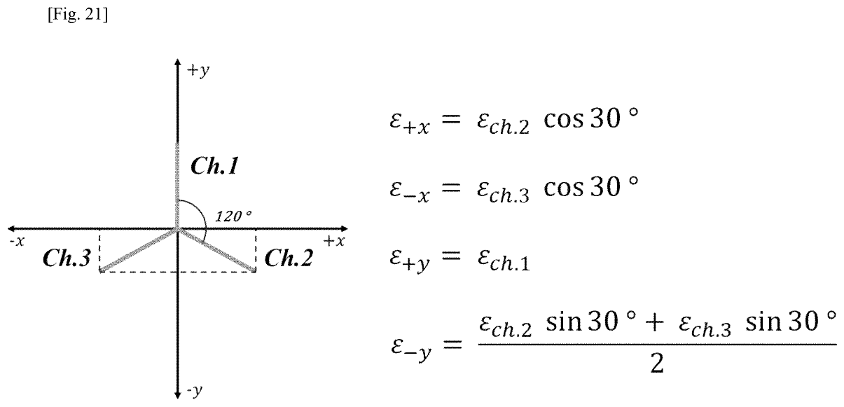

FIG. 20(a) is a graph drawn according to the coordinates of a multi-channel bladder sensor, and FIG. 20(b) is a graph converted to x-y coordinates by using the formula in FIG. 21.

FIG. 21 illustrates a concept of detecting the expansion direction of the bladder through a multi-channel bladder sensor according to an embodiment.

By tracking the relative expansion of each channel, it was assumed that when the resistance is R0, the relative expansion is 1. As a result, the volume of pig's bladder was rapidly expanding up to 300 mL in the −x direction, and thereafter, uniformly expanding in the +x and +y directions. It can be therefore identified that the bladder sensor can sufficiently track the direction of expansion depending on the volume of the bladder.

Meanwhile, in one embodiment, as described above, as for the effect of the pressure from the curvature of the bladder on the resistance of the bladder sensor, Formula 3 below may be used to define the error values between the actual strain and the calculated strain by using the resistance change rate and the gauge factor.

$$( 1 - \frac{L / {L}_{0}}{\frac{Δ R / {R}_{0}}{GF} + 1} ) \times 100 \% \qquad [ Equation 3 ]$$

In an embodiment, the error of the result with stick-shaped bladder sensor and the error of the result with multi-channel bladder sensor may be calculated using Formula 3 above. As a result, the error of the stick-shaped bladder sensor was calculated as 39.19% and the errors of the multi-channel bladder sensor were calculated as 27.02%, 3317%, and 19.69% respectively for each channel.

That is, it was experimentally identified that the curvature of the bladder wall affected the verification of the bladder sensor attached on the wall surface. Therefore, a multi-channel bladder sensor can be used to reduce the effect of the pressure from the curvature of the bladder on the resistance of the bladder sensor. In addition, it can be identified on the basis of the experimental results that the shorter the length of the bladder sensor, the less the effect of pressure from the curvature of the bladder on the resistance of the bladder sensor.

Then, on the basis of the multi-channel bladder sensor verified through the above-described verification methods, a bladder monitoring method for monitoring the bladder sensor based on a multi-channel strain bladder sensor is described.

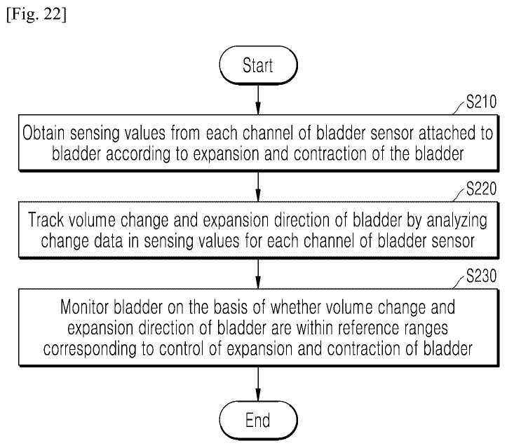

FIG. 22 is a flow chart for illustrating a bladder monitoring method based on a multi-channel strain bladder sensor according to an embodiment.

Referring to FIG. 22, in step S210, a processor 160 acquires sensing values from each channel of the bladder sensor attached to the bladder as the bladder expands and contracts.

In step S220, the processor 160 analyzes change data in the sensed values for each channel of the bladder sensor to track the volume change and expansion direction of the bladder.

Particularly, the processor 160 may measure the extended length of a base substrate of the bladder sensor on the basis of the resistance values of each channel of the bladder sensor, and calculate the length of expansion in the axial direction corresponding to each channel according to the extended length of each channel.

In step S230, the processor 160 may monitor the bladder, on the basis of whether the volume change and expansion direction of the bladder are within reference ranges corresponding to the control of the expansion and contraction of the bladder.

Particularly, the processor 160 may check whether the length of expansion in each expansion direction of the bladder sensor is within a predetermined expansion range, and may provide a notification for urination timing if the length of expansion in each expansion direction of the bladder sensor is within the predetermined expansion range.

In addition, the processor 160 may check whether after urination, the length of expansion in each expansion direction of the bladder sensor is contracted to the length when the bladder is in its normal state. Additionally, the processor 160 may determine incomplete urination if the length of expansion in each expansion direction of the bladder sensor is not contracted to the length when the bladder is in its normal state, and then provide a notification that additional urination is needed.

In other words, in an embodiment, a flexible bladder sensor created under optimal fabrication conditions can be used for bladder monitoring, and after verification of the bladder sensor, the bladder sensor can be applied in practice to perform monitoring.

Embodiments of the present disclosure described above may be implemented in the form of computer programs that may be executed through various components on a computer, and such computer programs may be recorded in a computer-readable medium. In this case, examples of the computer-readable media may include, but are not limited to: magnetic media such as hard disks, floppy disks, and magnetic tape; optical media such as CD-ROM disks and DVD-ROM disks; magneto-optical media such as floptical disks; and hardware devices that are specially configured to store and execute program instructions, such as ROM, RAM, and flash memory devices.

The computer programs may be those specially designed and constructed for the purposes of the present disclosure or they may be of the kind well known and available to those skilled in the art of computer software. Examples of program code include both machine code, such as that produced by a compiler, and higher level code that may be executed by the computer using an interpreter.

As used in the present disclosure (particularly in the appended claims), the term “the” and similar demonstrative terms include both singular and plural references. Also, it should be understood that any numerical range recited herein is intended to include all sub-ranges subsumed therein (unless expressly indicated otherwise) and accordingly, the disclosed numerical ranges include every individual value between the minimum and maximum values of the numerical ranges.

The order of individual steps in process claims according to the present disclosure does not imply that the steps must be performed in this order; rather, the steps may be performed in any suitable order, unless expressly indicated otherwise. The present disclosure is not necessarily limited to the order of operations given in the description. All examples described herein or terms indicative thereof (“for example,” etc.) used herein are merely to describe the present disclosure in greater detail. Therefore, it should be understood that the scope of the present disclosure is not limited to the exemplary embodiments described above or by the use of such terms unless limited by the appended claims. Also, it should be apparent to those skilled in the art that various modifications, combinations, and alternations may be made depending on design conditions and factors within the scope of the appended claims or equivalents thereto.

The present disclosure is thus not limited to the example embodiments described above, and rather is intended to include the following appended claims, and all modifications, equivalents, and alternatives falling within the spirit and scope of the following claims.

1. A multi-channel strain bladder sensor verification method for verifying a bladder sensor configured to be strained in multiple channels, the method comprising:
controlling the expansion and contraction of a measurement object serving as a bladder;
obtaining sensing values from each channel of a bladder sensor attached to the measurement object according to the controlling of the expansion and contraction of the measurement object;
tracking the volume change and expansion direction of the measurement object by analyzing change data in sensing values for each channel of the bladder sensor; and
verifying the bladder sensor on the basis of whether the volume change and expansion direction of the measurement object are within reference ranges corresponding to the control of the expansion and contraction of the measurement object,
wherein at least a part of each step is performed by a processor.

2. The method of claim 1, wherein the controlling of the expansion and contraction of the measurement object comprises controlling the inflow and outflow of a liquid with respect to the measurement object through one pair of inlet and outlet lines connected to allow the liquid to be injected into the measurement object,
the one pair of inlet and outlet lines comprising: a circulator allowing the liquid to circulate; a valve capable of being controlled to open and close, and a flow rate sensor, and
wherein the controlling of the inflow and outflow of the liquid with respect to the measurement object comprises driving the circulator and the valve through a driver on the basis of flow rate sensing values from the flow rate sensor, thereby controlling the inflow and outflow of the liquid with respect to the measurement object.

3. The method of claim 1, wherein the tracking of the volume change and expansion direction of the measurement object comprises:
measuring the extended length of a base substrate of the bladder sensor, on the basis of a resistance value for each channel of the bladder sensor; and
calculating the length of expansion in the axial direction corresponding to each channel according to the extended length of each channel, by using trigonometry, and
wherein the bladder sensor is a resistive sensor in which as the measurement object expands, the conductivity between coated electrical conductors decreases, resulting in an increase in resistance value.

4. The method of claim 3, wherein the verifying of the bladder sensor comprises:
injecting a predetermined amount of a liquid into the measurement object at the initial state, which is expanded corresponding to a reference bladder volume, to check whether the length of expansion in each expansion direction of the bladder sensor is within an expansion range corresponding to the amount of the liquid injected; and
allowing the injected liquid to flow out to check whether the length of expansion in each expansion direction of the bladder sensor is within the normal range based on the length of expansion at the initial state.

5. The method of claim 1, wherein the bladder sensor is configured to have a structure where a carbon nanotube (CNT) film and an AuCNT composite are deposited on an Ecoflex base substrate.

6. The method of claim 1, wherein the bladder sensor is configured as a three-channel sensor with channels spaced at intervals of 120 degrees.

7. The method of claim 1, wherein the bladder sensor is configured such that a gauge factor indicating a resistance change in response to the strain according to the strain characteristics of the bladder.

8. A multi-channel strain bladder sensor verification apparatus for verifying a bladder sensor configured to be strained in multiple channels, the apparatus comprising:
a memory; and
one processor connected to the memory and configured to execute computer-readable commands included in the memory,
wherein the at least one processor is configured to perform the operations comprising:
controlling the expansion and contraction of a measurement object serving as a bladder;
obtaining sensing values from each channel of a bladder sensor attached to the measurement object according to the controlling of the expansion and contraction of the measurement object;
tracking the volume change and expansion direction of the measurement object by analyzing change data in sensing values for each channel of the bladder sensor; and
verifying the bladder sensor on the basis of whether the volume change and expansion direction of the measurement object are within reference ranges corresponding to the control of the expansion and contraction of the measurement object.

9. The apparatus of claim 8, wherein the controlling of the expansion and contraction of the measurement object comprises controlling the inflow and outflow of a liquid with respect to the measurement object through one pair of inlet and outlet lines connected to allow the liquid to be injected into the measurement object,
the one pair of inlet and outlet lines comprising: a circulator allowing the liquid to circulate; a valve capable of being controlled to open and close, and a flow rate sensor, and
wherein the controlling of the inflow and outflow of the liquid with respect to the measurement object comprises driving the circulator and the valve through a driver on the basis of flow rate sensing values from the flow rate sensor, thereby controlling the inflow and outflow of the liquid with respect to the measurement object.

10. The apparatus of claim 8, wherein the tracking of the volume change and expansion direction of the measurement object comprises:
measuring the extended length of a base substrate of the bladder sensor, on the basis of a resistance value for each channel of the bladder sensor; and
calculating the length of expansion in the axial direction corresponding to each channel according to the extended length of each channel, by using trigonometry, and
wherein the bladder sensor is a resistive sensor in which as the measurement object expands, the conductivity between coated electrical conductors decreases, resulting in an increase in resistance value.

11. The apparatus of claim 10, wherein the verifying of the bladder sensor comprises:
injecting a predetermined amount of a liquid into the measurement object at the initial state, which is expanded corresponding to a reference bladder volume, to check whether the length of expansion in each expansion direction of the bladder sensor is within an expansion range corresponding to the amount of the liquid injected; and
allowing the injected liquid to flow out to check whether the length of expansion in each expansion direction of the bladder sensor is within the normal range based on the length of expansion at the initial state.

12. The apparatus of claim 8, wherein the bladder sensor is configured to have a structure where a carbon nanotube (CNT) film and an AuCNT composite are deposited on an Ecoflex base substrate.

13. The apparatus of claim 8, wherein the bladder sensor is configured as a three-channel sensor with channels spaced at intervals of 120 degrees.

14. The apparatus of claim 8, wherein the bladder sensor is configured such that a gauge factor indicating a resistance change in response to the strain according to the strain characteristics of the bladder.

15. A bladder monitoring method, based on a multi-channel strain bladder sensor, for monitoring the bladder by using a bladder sensor configured to be strained in multiple channels, the method comprising:
obtaining sensing values from each channel of a bladder sensor attached to the bladder according to the expansion and contraction of the bladder;
tracking the volume change and expansion direction of the bladder by analyzing change data in sensing values for each channel of the bladder sensor; and
monitoring the bladder on the basis of whether the volume change and expansion direction of the bladder are within reference ranges corresponding to the control of the expansion and contraction of the bladder,
wherein at least a part of each step is performed by a processor.

16. The method of claim 15, wherein the tracking of the volume change and expansion direction of the bladder comprises:
measuring the extended length of a base substrate of the bladder sensor, on the basis of a resistance value for each channel of the bladder sensor; and
calculating the length of expansion in the axial direction corresponding to each channel according to the extended length of each channel, by using trigonometry, and
wherein the bladder sensor is a resistive sensor in which as the bladder expands, the conductivity between coated electrical conductors decreases, resulting in an increase in resistance value.

17. The method of claim 16, wherein the monitoring of the bladder comprises:
checking whether the length of expansion in each expansion direction of the bladder sensor is within a predetermined expansion range; and
providing a notification for urination timing if the expansion length in each expansion direction of the bladder sensor is within the predetermined expansion range.

18. The method of claim 17, wherein the monitoring of the bladder comprises:
checking whether after urination, the expansion length in each expansion direction of the bladder sensor is contracted to the length when the bladder is in its normal state; and
determining incomplete urination if the expansion length in each expansion direction of the bladder sensor is not contracted to the length when the bladder is in its normal state, and then providing a notification that additional urination is needed.

19. A bladder monitoring apparatus based on a multi-channel strain bladder sensor, for monitoring the bladder, by using a bladder sensor configured to be strained in multiple channels, the apparatus comprising:
a memory; and
one processor connected to the memory and configured to execute computer-readable commands included in the memory,
wherein the at least one processor is configured to perform the operations comprising:
obtaining sensing values from each channel of a bladder sensor attached to the bladder according to the expansion and contraction of the bladder;
tracking the volume change and expansion direction of the measurement object by analyzing change data in sensing values for each channel of the bladder sensor; and
monitoring the bladder on the basis of whether the volume change and expansion direction of the bladder are within reference ranges corresponding to the control of the expansion and contraction of the measurement object.

20. The apparatus of claim 19, wherein the tracking of the volume change and expansion direction of the bladder comprises:
measuring the extended length of a base substrate of the bladder sensor, on the basis of a resistance value for each channel of the bladder sensor; and
calculating the length of expansion in the axial direction corresponding to each channel according to the extended length of each channel, by using trigonometry, and
wherein the bladder sensor is a resistive sensor wherein as the bladder expands, the conductivity between coated electrical conductors decreases, resulting in an increase in resistance value.

21. The apparatus of claim 20, wherein the monitoring of the bladder comprises:
checking whether the length of expansion in each expansion direction of the bladder sensor is within a predetermined expansion range; and
providing a notification for urination timing if the expansion length in each expansion direction of the bladder sensor is within the predetermined expansion range.

22. The apparatus of claim 21, wherein the monitoring of the bladder comprises:
checking whether after urination, the expansion length in each expansion direction of the bladder sensor is contracted to the length when the bladder is in its normal state; and
determining incomplete urination if the expansion length in each expansion direction of the bladder sensor is not contracted to the length when the bladder is in its normal state, and then providing a notification that additional urination is needed.
